(19) United States
(12) Patent Application Publication
(10) Pub. No.: US 2022/0193042 A1
(43) Pub. Date: Jun. 23, 2022

(54) NITAZOXANIDE AND THIAZOLIDES FOR USE IN THE TREATMENT OF DISEASES ASSOCIATED WITH OXIDATIVE STRESS

(71) Applicant: GENFIT, Loos (FR)

(72) Inventors: Nicolas STANKOVIC-VALENTIN, Mouscron (BE); Corinne FOUCART, La Madeleine (FR); Peggy PARROCHE, Loos (FR); Robert WALCZAK, Lille (FR)

(21) Appl. No.: 17/599,310

(22) PCT Filed: Apr. 9, 2020
(86) PCT No.: PCT/EP2020/060287

§ 371 Date: Sep. 28, 2021

(30) Foreign Application Priority Data
Apr. 12, 2019 (EP) 19305480.6

(51) Int. Cl. A61K 31/426; A61P 39/06

(52) U.S. Cl. CPC A61K 31/426; A61P 39/06

(57) ABSTRACT

The present invention relates to novel uses of nitazoxanide, or analogues thereof.

The present invention relates to novel uses of nitazoxanide, or analogues thereof.

[2-[(5-nitro-1,3-thiazol-2-yl)carbamoyl]phenyl]ethanoate (or nitazoxanide, or NTZ), first described in 1975 (Rossignol and Cavier 1975) is a medicament authorized in the United States for the treatment of diarrhea caused by the protozoan parasites Crystosporidium parvum and Giardia intestinalis. NTZ has also been commercialized in Latin America and in India where it is indicated for treating a broad spectrum of intestinal parasitic infections. The proposed mechanism of action by which NTZ exerts its antiparasitic activity is through the inhibition of pyruvate:ferredoxin oxidoreductase (PFOR) enzyme-dependent electron transfer reactions that are essential for anaerobic metabolism (Hoffman, Sisson et al. 2007). NTZ also exhibited activity against Mycobacterium tuberculosis, which does not possess a homolog of PFOR, thus suggesting an alternative mechanism of action. Indeed, the authors showed that NTZ can also act as an uncoupler disrupting membrane potential and intra-organism pH homeostasis (de Carvalho, Darby et al. 2011).

The pharmacological effects of NTZ are not restricted to its antiparasitic or antibacterial activities and in recent years, several studies revealed that NTZ can also confer antiviral activity by interfering with the viral replication by diverse ways including a blockade in the maturation of hemagglutinin (influenza) or VP7 (rotavirus) proteins, or the activation of the protein PKR involved in the innate immune response (for a review, see Rossignol 2014). NTZ was also shown to have anticancer properties by interfering with crucial metabolic and pro-death signalling pathways (Di Santo and Ehrisman 2014).

NTZ was also recently shown by the present Applicant to have antifibrotic properties (WO2017178172) and is currently evaluated for its effect on a population with NASH-induced stage 2 or 3 fibrosis.

The inventors herein show that NTZ has antioxidant properties, which opens new therapeutic opportunities.

The invention stems from the surprising observation made by the inventors that NTZ activates the expression of different glutathione S-transferase (GST) genes. GSTs are a family of enzymes that play an important role in detoxification by catalysing the conjugation of many hydrophobic and electrophilic compounds with reduced glutathione. GSTs have a particular role in protecting cells from reactive oxygen species and the products of peroxidation. Their activation can thus advantageously be implemented for protecting cells, tissues and organs against oxidative stress.

Complementary analyses of the hepatic transcriptomic signature induced by NTZ revealed an enrichment of a subset of genes (including GSTs enzymes) under the control of Nrf2, a transcriptional master regulator of intracellular redox homeostasis. In vitro analyses confirmed at the functional level the capacity of TZ (the active metabolite of NTZ) to induce Nrf2-ARE signalling pathway. In addition, it was demonstrated that TZ is able to preserve the GSH (reduced Glutathione) pool, the most abundant antioxidant in the liver, in human hepatocytes under oxidative stress. Altogether, these data demonstrate the unexpected antioxidant capacities of NTZ.

An aspect of the invention thus relates to a compound of formula (I) as defined below, which includes NTZ and analogues thereof, or a pharmaceutically acceptable salt of a compound of formula (I), for use as an antioxidant. In a particular embodiment the compound of formula (I) or a pharmaceutically acceptable salt thereof is used for its hepatic antioxidant properties.

Another aspect of the invention relates to a compound of formula (I) or a pharmaceutically acceptable salt thereof, for use in a method for treating a disease in which oxidative stress is involved. Diseases in which oxidative stress is involved are commonly known to those skilled in the art who can refer, among many other sources to de Araujo et al. (de AraGjo, Martins et al. 2016). For example, subjects who can benefit from the invention include, without limitation, those suffering from neurological disorders such as central nervous system disorders, metabolic conditions, cardiovascular diseases, cataract, atherosclerosis, ischemia such as myocardial ischemia, ischemic brain damage, lung ischemia-reperfusion injury, scleroderma and stroke, inflammation such as inflammatory bowel disease, rheumatoid arthritis, respiratory diseases, autoimmune diseases, kidney diseases and skin conditions.

The term “treatment” or “treating” refers to the curative or preventive treatment of a disease in a subject in need thereof. The treatment involves the administration of the compound of the invention to a subject having a declared disease, to prevent, cure, delay, reverse, or slow down the progression of the disease, improving thereby the condition of the subject. The invention thus also relates to a compound of formula (I), or a pharmaceutically acceptable salt thereof, for use in a method to prevent, cure delay, reverse or slow down the progression of the disease. The compound of the invention can also be administered to a subject that is healthy or at risk of developing a disease. The subject to be treated is a mammal, preferably a human. The subject to be treated according to the invention can be selected on the basis of several criteria associated to the specific disease the treatment of which is sought such as previous drug treatments, associated pathologies, genotype, exposure to risk factors, viral infection, as well as on the basis of the detection of any biomarker relevant to the disease.

In addition, the invention relates to a compound of formula (I) or a pharmaceutically acceptable salt thereof, for use in a method for treating the oxidative stress associated to a disease, in particular a disease selected in the group consisting of neurological disorders such as central nervous system disorders, metabolic conditions, cardiovascular diseases, cataract, atherosclerosis, ischemia such as myocardial ischemia, ischemic brain damage, lung ischemia-reperfusion injury, scleroderma and stroke, inflammation such as inflammatory bowel disease, rheumatoid arthritis, respiratory diseases, autoimmune diseases, liver diseases, kidney diseases, skin conditions, infections and cancers.

Neurological disorders include, without limitation, Alzheimer's disease, Parkinson's disease, Huntington's disease, tardive dyskinesia, epilepsy and acute diseases of the central nervous system such as spinal cord injuries and/or brain trauma.

Metabolic conditions include, without limitation, obesity, insulin resistance, dyslipidemia, impaired glucose tolerance, high blood pressure, atherosclerosis and diabetes, such as type 1 or type 2 diabetes. Metabolic conditions also include the metabolic syndrome.

In a particular embodiment, the compounds of formula (I) or a pharmaceutically acceptable salt thereof are used in a method for treating infection-induced oxidative stress, such as virus-induced oxidation stress, in particular human immunodeficiency virus-induced oxidative stress, influenza virus-induced oxidative stress, HBV-induced oxidative stress, hepatitis C virus-induced oxidative stress, encephalomyocarditis virus-induced oxidative stress, respiratory syncytial virus-induced oxidative stress and dengue virus-induced oxidative stress.

The invention further relates to a compound of formula (I) or a pharmaceutically acceptable salt thereof, for use in a method for treating the oxidative stress associated to liver disorders. Thus, the compound of formula (I) may be used in a method for the treatment of the oxidative stress associated to a liver disorder. In particular, the subject to be treated can have cirrhosis, non-alcoholic fatty liver disease (NAFLD), NAFLD with liver fibrosis, non-alcoholic steatohepatitis (NASH), NASH with liver fibrosis or NASH with liver cirrhosis. The invention therefore also relates to a compound of formula (I) or a pharmaceutically acceptable salt thereof, for use in a method for treating the oxidative stress associated to cirrhosis, the oxidative stress associated to NAFLD, the oxidative stress associated to NAFLD with liver fibrosis, the oxidative stress associated to NASH, the oxidative stress associated to NASH with liver fibrosis, or the oxidative stress associated to NASH with liver cirrhosis. In another particular embodiment, the subject to be treated has NAFLD, NAFLD with liver fibrosis, NASH or NASH with liver fibrosis. Thus, in a particular embodiment of the invention, a compound of formula (I), or a pharmaceutically acceptable salt thereof, is used in a method for treating oxidative stress associated to NAFLD, oxidative stress associated to NAFLD with liver fibrosis, oxidative stress associated to NASH or oxidative stress associated to NASH with liver fibrosis.

The invention further relates to a method for the treatment of the oxidative stress associated to a liver disorder, wherein the method comprises the administration of a therapeutically effective amount of a compound of formula (I) or a pharmaceutically acceptable salt thereof to a subject in need of a treatment of a liver disorder. In a particular embodiment of the method of the invention, the subject has cirrhosis, non-alcoholic fatty liver disease (NAFLD), NAFLD with liver fibrosis, non-alcoholic steatohepatitis (NASH), NASH with liver fibrosis or NASH with liver cirrhosis.

In a particular embodiment, the compounds of formula (I) or a pharmaceutically acceptable salt thereof are used in a method for treating oxidative stress associated to cancer, in particular liver cancer, more particularly hepatocellular carcinoma (HCC).

The invention further relates to a method for the treatment of the oxidative stress associated to a cancer, in particular a liver cancer, such as a hepatocellular carcinoma, wherein the method comprises the administration of a therapeutically effective amount of a compound of formula (I) or a pharmaceutically acceptable salt thereof to a subject in need thereof.

The compounds used in the present invention are of formula (I):

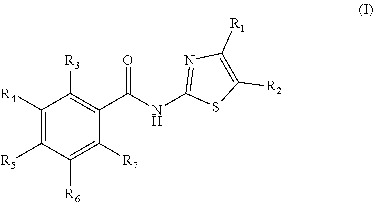

in which:
R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group (N02), an amino group (NH2), a (C1-C6)alkylamino group, an amido group, a (C1-C6)alkylamido group or a (C1-C6)dialkylamido group;
R2 represents a hydrogen atom, a deuterium atom, a N02 group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH₂ group or a (C1-C6)alkylamino group;
or R1 and R2, together with the carbon atoms to which they are attached, form a substituted or unsubstituted 5- to 8-membered cycloalkyl, heterocyclic or aryl group;
R3, R4, R5, R6, and R7, identical or different, represent a hydrogen atom, a deuterium atom, a halogen atom, a hydroxyl group, a (C1-C6)alkylcarbonyl group, an (C1-C6)alkyl group, an (C1-C6)alkyloxy group, an (C1-C6)alkylthio group, an (C1-C6)alkylcarbonyloxy group, an (C6-C14)aryloxy group, a (C6-C14)aryl group, a heterocyclic group, a (C3-C14)cycloalkyl group, a N02 group, a sulfonylaminoalkyle group, an NH2 group, an amino(C1-C6)alkyl group, an (C1-C6)alkylcarbonylamino group, a carboxylic group, a carboxylate group, or a R9 group;
R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

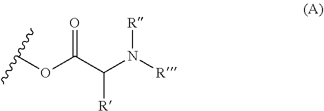

wherein R′ represents an (C1-C6)alkyl group, an (C2-C6)alkenyl group, an (C2-C6)alkynyl group, a (C3-C14)cycloalkyl group, (C3-C14)cycloalkylalkyl group, a (C3-C14)cycloalkyl(C2-C6)alkenyl group, a (C3-C14)cycloalkenyl group, a (C3-C14)cycloalkenyl(C1-C6)alkyl group, a (C3-C14)cycloalkenyl(C2-C6)alkenyl group or a (C3-C14)cycloalkenyl(C2-C6)alkynyl group; wherein R″ and R′″, independently, represent a hydrogen atom, an (C1-C6)alkyl group, or a nitrogen protecting group; and
R8 represents a hydrogen atom, a deuterium atom, a glucuronidyl group, or a

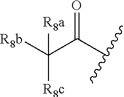

group wherein, R8a, R8b and R8c, identical or different, represent a hydrogen atom or a deuterium atom.

In a further particular embodiment, the compound of formula (I) is as follows:
R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a NO₂ group, a NH2 group, a (C1-C6)alkylamino group, an amido group, a (C1-C6)alkylamido group or a (C1-C6)dialkylamido group;
R2 represents a hydrogen atom, a deuterium atom, a N02 group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH₂ group or a (C1-C6)alkylamino group;
or R1 and R2, together with the carbon atoms to which they are attached, form a substituted or unsubstituted 5- to 8-membered cycloalkyl, heterocyclic or aryl group;
R3 represents a hydrogen atom, a deuterium atom, a halogen atom, a O—R8 group, a (C1-C6)alkylcarbonyl group, an (C1-C6)alkyl group, an (C1-C6)alkyloxy group, an (C1-C6)alkylthio group, an (C1-C6)alkylcarbonyloxy group, an (C6-C14)aryloxy group, a (C6-C14)aryl group, a heterocyclic group, a (C3-C14)cycloalkyl group a N02, a sulfonylaminoalkyle group, an NH2 group, an amino(C1-C6)alkyl group, an (C1-C6)alkylcarbonylamino group, a carboxylic group, a carboxylate group, an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

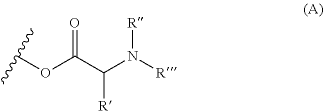

wherein R′ represents an (C1-C6)alkyl group, an (C2-C6)alkenyl group, an (C2-C6)alkynyl group, a (C3-C14)cycloalkyl group, (C3-C14)cycloalkylalkyl group, a (C3-C14)cycloalkyl(C2-C6)alkenyl group, a (C3-C14)cycloalkenyl group, a (C3-C14)cycloalkenyl(C1-C6)alkyl group, a (C3-C14)cycloalkenyl(C2-C6)alkenyl group or a (C3-C14)cycloalkenyl(C2-C6)alkynyl group; wherein R″ and R′″, independently, represent hydrogen atom, an (C1-C6)alkyl group, or a nitrogen protecting group;
R8 represents a hydrogen atom, a deuterium atom, or a

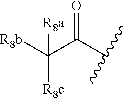

group wherein, R8a, R8b and R8c, identical or different, represent a hydrogen atom or a deuterium atom; and
R4, R5, R6, and R7, identical or different, represent a hydrogen atom, a deuterium atom, a halogen atom, a hydroxyl group, an (C1-C6)alkylcarbonyl group, an (C1-C6)alkyl group, an (C1-C6)alkyloxy group, an (C1-C6)alkylthio group, an (C1-C6)alkylcarbonyloxy group, an (C6-C14)aryloxy group, an (C6-C14)aryl group, a heterocyclic group, a (C3-C14)cycloalkyl group, a N02, a sulfonylamino(C1-C6)alkyl group, an NH2 group, an amino(C1-C6)alkyl group, an (C1-C6)alkylcarbonylamino group, a carboxylic group, a carboxylate group, an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

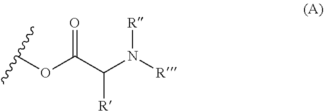

wherein R′ represents an (C1-C6)alkyl group, an (C2-C6)alkenyl group, an (C2-C6)alkynyl group, a (C3-C14)cycloalkyl group, (C3-C14)cycloalkyl(C1-C6)alkyl group, a (C3-C14)cycloalkyl(C1-C6)alkenyl group, a (C3-C14)cycloalkenyl group, a (C3-C14)cycloakenyl(C1-C6)alkyl group, a (C3-C14)cycloalkenyl(C2-C6)alkenyl group, a (C3-C14)cycloalkenyl(C2-C6)alkynyl group; R″ and R′″, independently, represent a hydrogen atom, an (C1-C6)alkyl group, or a nitrogen protecting group.

In a particular embodiment, in the compound of formula (I) of the present invention: an alkyl group may be a substituted or unsubstituted (C1-C6)alkyl group, in particular a substituted or unsubstituted (C1-C4)alkyl group;

an alkynyl group may be a substituted or unsubstituted (C2-C6)alkynyl group;

a cycloalkyl group may be a substituted or unsubstituted (C3-C14)cycloalkyl group

an alkyloxy group may be a substituted or unsubstituted (C1-C6)alkyloxy group, such as a

substituted or unsubstituted (C1-C4)alkyloxy group;

an alkylthio group may be a substituted or unsubstituted (C1-C6)alkylthio group, such as a substituted or unsubstituted (C1-C4)alkylthio group;

an alkylamino group may be a (C1-C6)alkylamino group, such as a (C1-C4)alkylamino group;

a dialkylamino group may be a (C1-C6)dialkylamino group, such as a (C1-C4)dialkylamino group;

an aryl group may be a substituted or unsubstituted (C6-C14)aryl group, such as a substituted or unsubstituted (C6-C14)aryl group;

a heterocyclic group may be a substituted or unsubstituted heterocycloalkyl or heteroaryl group.

Nitrogen protecting groups are well known to those skilled in the art, such as those described in the literature, as, for example, in the book “Greene's Protective Groups in Organic Synthesis” (Wuts and Greene 2007).

In a specific embodiment, the compound of formula (I) is a compound of formula (II):

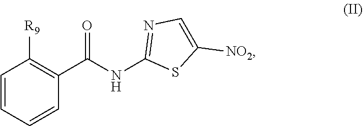

in which R9 represents a hydrogen atom, a deuterium atom, a O—R8 group (R8 being as defined above), or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

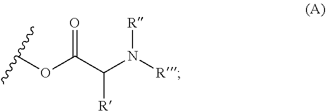

wherein R′ represents an (C1-C6)alkyl group, an (C2-C6)alkenyl group, an (C2-C6)alkynyl group, a (C3-C14)cycloalkyl group, (C3-C14)cycloalkyl(C1-C6)alkyl group, a (C3-C14)cycloalkyl(C1-C6)alkenyl group, a (C3-C14)cycloalkenyl group, a (C3-C14)cycloakenyl(C1-C6)alkyl group, a (C3-C14)cycloalkenyl(C2-C6)alkenyl group or a (C3-C14)cycloalkenyl(C2-C6)alkynyl group; wherein R″ and R′″, independently, represent a hydrogen atom, an (C1-C6)alkyl group, or a nitrogen protecting group.

In a particular embodiment, the compound of formula (I) is selected from:

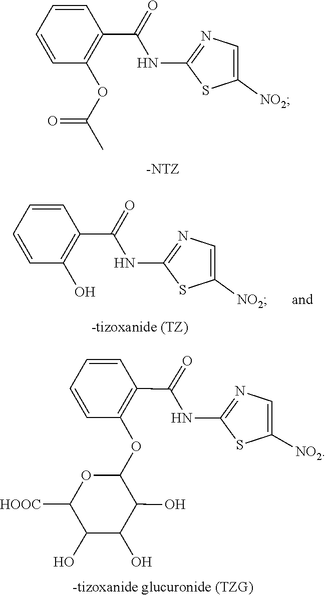 [SMILES: CC(=O)Oc1ccccc1C(=O)Nc1ncc([N+](=O)[O-])s1 ; O=C(Nc1ncc([N+](=O)[O-])s1)c1ccccc1O ; O=C(Nc1ncc([N+](=O)[O-])s1)c1ccccc1OC1OC(C(=O)O)C(O)C(O)C1O]

In another embodiment, the compound of formula (II) is such that
R8a, R8b and R8c, identical or different, represent a hydrogen atom or a deuterium atom; and/or
R1, R3, R4, R5, and R6, identical or different, represent a hydrogen atom or a deuterium atom with the proviso that R1, R2, R8a, R8b, R8c, R3, R4, R5, and R6 are not simultaneously a hydrogen atom.

In a particular embodiment, the compound of formula (I) is [(5-nitro-1,3-thiazol-2-yl)carbamoyl]phenyl (d3)ethanoate, 2-[(5-nitro-1,3-thiazol-2-yl)carbamoyl]phenyl (d2) ethanoate; or 2-[(5-nitro-1,3-thiazol-2-yl)carbamoyl]phenyl (d1) ethanoate.

In another particular embodiment, the compound of formula (I) is 2-(5-nitrothiazol-2-ylcarbamoyl)phenyl 2-amino-3,3-dimethylbutanoate, in particular (S)-2-(5-nitrothiazol-2-ylcarbamoyl)phenyl 2-amino-3,3-dimethylbutanoate, or a pharmaceutically acceptable salt thereof such as its hydrochloride salt (RM5061) of formula:

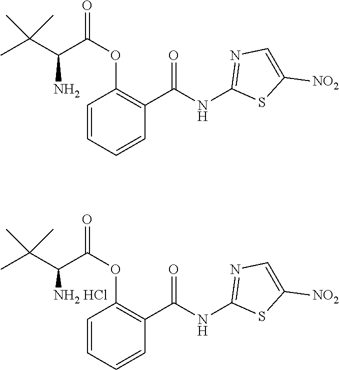 [SMILES: CC(C)(C)[C@H](N)C(=O)Oc1ccccc1C(=O)Nc1ncc([N+](=O)[O-])s1 ; CC(C)(C)[C@H](N)C(=O)Oc1ccccc1C(=O)Nc1ncc([N+](=O)[O-])s1 ; Cl]

In another particular embodiment, the compound of formula (I) is 2-(5-nitrothiazol-2-ylcarbamoyl)phenyl 2-amino-3-methylpentanoate, in particular (2S,3S)-2-(5-nitrothiazol-2-ylcarbamoyl)phenyl 2-amino-3-methylpentanoate, or a pharmaceutically acceptable salt thereof such as its hydrochloride salt (RM5066) of formula:

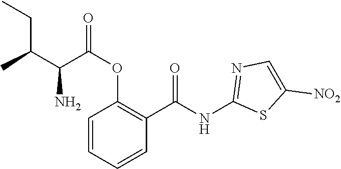 [SMILES: CC[C@H](C)[C@H](N)C(=O)Oc1ccccc1C(=O)Nc1ncc([N+](=O)[O-])s1]

In another particular embodiment, the compound of formula (I) is 2-(5-chlorothiazol-2-ylcarbamoyl)phenyl 2-amino-3,3-dimethylbutanoate, in particular (S)-2-(5-chlorothiazol-2-ylcarbamoyl)phenyl 2-amino-3,3-dimethylbutanoate, or a pharmaceutically acceptable salt thereof such as its hydrochloride salt (RM5064) of formula:

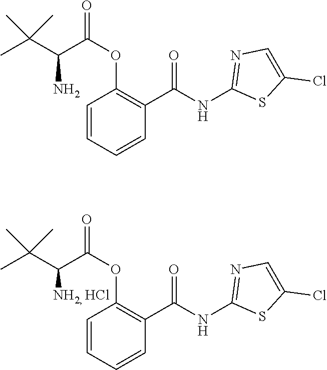 [SMILES: CC(C)(C)[C@H](N)C(=O)Oc1ccccc1C(=O)Nc1ncc(Cl)s1 ; CC(C)(C)[C@H](N)C(=O)Oc1ccccc1C(=O)Nc1ncc(Cl)s1 ; Cl]

In another particular embodiment, the compound of formula (I) is -2-(5-chlorothiazol-2-ylcarbamoyl)phenyl 2-amino-3-methylpentanoate, in particular (2S,3S)-2-(5-chlorothiazol-2-ylcarbamoyl)phenyl 2-amino-3-methylpentanoate, or a pharmaceutically acceptable salt thereof such as its hydrochloride salt (RM5065) of formula:

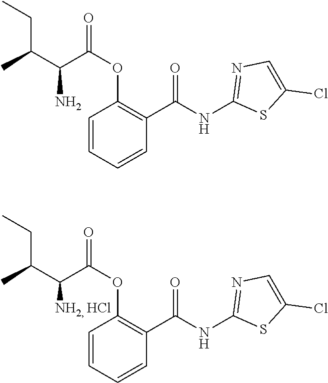 [SMILES: CC[C@H](C)[C@H](N)C(=O)Oc1ccccc1C(=O)Nc1ncc(Cl)s1 ; CC[C@H](C)[C@H](N)C(=O)Oc1ccccc1C(=O)Nc1ncc(Cl)s1 ; Cl]

In a particular embodiment, the compound of formula (I) is NTZ, TZ, TZG or a pharmaceutically acceptable salt thereof. In a further particular embodiment, the compound of formula (I) is NTZ or TZ or a pharmaceutically acceptable salt thereof. In a preferred embodiment, the compound of formula (I) is NTZ or a pharmaceutically acceptable salt thereof.

In a particular embodiment, the invention relates to a method for the treatment of the oxidative stress associated to a liver disorder, wherein the method comprises the administration of a therapeutically effective amount of NTZ and/or TZ or a pharmaceutically acceptable salt thereof to a subject in need of a treatment of a liver disorder. In a particular embodiment, the subject is administered with NTZ or a pharmaceutically acceptable salt thereof.

In a further particular embodiment, the invention relates to a method for the treatment of the oxidative stress associated to liver fibrosis, wherein the method comprises the administration of a therapeutically effective amount of NTZ and/or TZ or a pharmaceutically acceptable salt thereof to a subject in need of a treatment of liver fibrosis. In a particular embodiment, the subject is administered with NTZ or a pharmaceutically acceptable salt thereof.

In another particular embodiment, the invention relates to a method for the treatment of the oxidative stress associated to cirrhosis, wherein the method comprises the administration of a therapeutically effective amount of NTZ and/or TZ or a pharmaceutically acceptable salt thereof to a subject in need of a treatment of cirrhosis. In a particular embodiment, the subject is administered with NTZ or a pharmaceutically acceptable salt thereof.

In another particular embodiment, the invention relates to a method for the treatment of the oxidative stress associated to NAFLD, wherein the method comprises the administration of a therapeutically effective amount of NTZ and/or TZ or a pharmaceutically acceptable salt thereof to a subject in need of a treatment of NAFLD. In a particular embodiment, the subject is administered with NTZ or a pharmaceutically acceptable salt thereof.

In another particular embodiment, the invention relates to a method for the treatment of the oxidative stress associated to NAFLD with liver fibrosis, wherein the method comprises the administration of a therapeutically effective amount of NTZ and/or TZ or a pharmaceutically acceptable salt thereof to a subject in need of a treatment of NAFLD with liver fibrosis. In a particular embodiment, the subject is administered with NTZ or a pharmaceutically acceptable salt thereof.

In a further particular embodiment, the invention relates to a method for the treatment of the oxidative stress associated to NASH, wherein the method comprises the administration of a therapeutically effective amount of NTZ and/or TZ or a pharmaceutically acceptable salt thereof to a subject in need of a treatment of NASH. In a particular embodiment, the subject is administered with NTZ or a pharmaceutically acceptable salt thereof.

In a further particular embodiment, the invention relates to a method for the treatment of the oxidative stress associated to NASH with liver fibrosis, wherein the method comprises the administration of a therapeutically effective amount of NTZ and/or TZ or a pharmaceutically acceptable salt thereof to a subject in need of a treatment of NASH with liver fibrosis. In a particular embodiment, the subject is administered with NTZ or a pharmaceutically acceptable salt thereof.

Synthesis of NTZ or analogues can be for example carried out as described in (Rossignol and Cavier 1975), or by any other way of synthesis known by a person skilled in the art.

The compound of formula (I) can be included in a pharmaceutical composition, with a pharmaceutically acceptable carrier. These pharmaceutical compositions can also comprise one or several excipients or vehicles, acceptable within a pharmaceutical context (e.g. saline solutions, physiological solutions, isotonic solutions, etc., compatible with pharmaceutical usage and well-known by one of ordinary skill in the art). These compositions can also comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. The compositions can be for enteral or parenteral administration. For example, the compound of formula (I) can be formulated for oral, intravascular (e.g. intravenous or intra-arterial), intramuscular, intraperitoneal, subcutaneous, transdermal or nasal administration. The composition can be a solid or liquid dosage form. Illustrative formulations include, without limitation, injectable suspensions, suspensions for oral ingestion, gels, oils, ointments, pills, tablets, suppositories, powders, gel caps, capsules, aerosols, ointments, creams, patches or means of galenic forms or devices assuring a prolonged and/or slow release. For this kind of formulation, agents such as cellulose, carbonates or starches can be advantageously used.

The compound of formula (I) can be formulated as a pharmaceutically acceptable salt, particularly acid or base salt compatible with pharmaceutical use. Salts of compounds of formula (I) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound. According to a specific embodiment, the composition of the invention comprises at least one compound of the formula (I) as active ingredients, together with acceptable excipients. As an example the composition of the invention comprises a combination of two compounds of formula (I), NTZ and TZ, as active ingredients.

The frequency and/or dose relative to the administration can be adapted by one of ordinary skill in the art, in function of the subject to be treated, the pathology, the form of administration, etc. Typically, the compound of formula (I), in particular NTZ or a pharmaceutically acceptable salt thereof can be administered at a dose comprised between 0.01 mg/day to 4000 mg/day, such as from 50 mg/day to 2000 mg/day, and particularly from 100 mg/day to 1000 mg/day, more particularly from 500 mg/day to 1 000 mg/day.

In another preferred embodiment, the compound of formula (I), preferably NTZ or a pharmaceutically acceptable salt thereof, is administered in the form of a pill or tablet intended for an oral ingestion. In another particular embodiment, the compound of formula (1), preferably NTZ or a pharmaceutically acceptable salt thereof, is administered in the form of a suspension for an oral ingestion.

In a further aspect, the invention relates to a method for the treatment of a disease, comprising the administration of NTZ or a pharmaceutical salt thereof, wherein NTZ is administered at a dose comprised between 500 mg/day and 1000 mg/day, wherein the disease is selected in the group consisting of Alzheimer's disease, Parkinson's disease, Huntington's disease, tardive dyskinesia, epilepsy, acute diseases of the central nervous system such as spinal cord injuries and/or brain trauma, obesity, insulin resistance, dyslipidemia, impaired glucose tolerance, high blood pressure, atherosclerosis and diabetes, such as type 1 or type 2 diabetes, metabolic syndrome, human immunodeficiency virus-induced oxidative stress, influenza virus-induced oxidative stress, HBV-induced oxidative stress, hepatitis C virus-induced oxidative stress, encephalomyocarditis virus-induced oxidative stress, respiratory syncytial virus-induced oxidative stress, dengue virus-induced oxidative stress, cirrhosis-associated oxidative stress, NAFLD-associated oxidative stress, NAFLD-associated oxidative stress with liver fibrosis, NASH-associated oxidative stress, NASH-associated oxidative stress with liver fibrosis, myocardial ischemia, ischemic brain damage, lung ischemia-reperfusion injury, scleroderma, stroke, inflammatory bowel disease and rheumatoid arthritis.

In a further aspect, the invention relates to a method for the treatment of a disease, comprising the administration of NTZ or a pharmaceutical salt thereof, wherein NTZ is administered at a dose comprised between 500 mg/day and 1000 mg/day, wherein the disease is selected in the group consisting of Alzheimer's disease, Parkinson's disease, Huntington's disease, tardive dyskinesia, epilepsy, acute diseases of the central nervous system such as spinal cord injuries and/or brain trauma, obesity, insulin resistance, dyslipidemia, impaired glucose tolerance, high blood pressure, atherosclerosis and diabetes, such as type 1 or type 2 diabetes, metabolic syndrome, human immunodeficiency virus-induced oxidative stress, influenza virus-induced oxidative stress, HBV-induced oxidative stress, hepatitis C virus-induced oxidative stress, encephalomyocarditis virus-induced oxidative stress, respiratory syncytial virus-induced oxidative stress, dengue virus-induced oxidative stress, NAFLD-associated oxidative stress, NAFLD-associated oxidative stress with liver fibrosis, NASH-associated oxidative stress, NASH-associated oxidative stress with liver fibrosis, myocardial ischemia, ischemic brain damage, lung ischemia-reperfusion injury, scleroderma, stroke, inflammatory bowel disease and rheumatoid arthritis.

Administration can be performed daily or even several times per day, if necessary. The duration of the treatment will depend on the specific disease to be treated. For example, the administration can be performed during one or several days, such as during at least one day, at least two days, at least three days, at least four days, at least five days, at six two days or at least seven days. Alternatively, the administration can be performed for at least one week, at least two weeks, at least four weeks. For chronic diseases, administration can be considered for more than four weeks, such as for at least one month, two months, three months, four months, five months, six months or more than six months, such as for at least one year or several years. In some cases, the combination product of the invention can be administered during the lifetime of the subject.

The invention is further described with reference to the following, non-limiting, examples.

### DESCRIPTION OF THE FIGURES

FIG. 1: chronic oral administration of NTZ contributes to antioxidant defence mechanisms.

6 week-old C57BL/6 mice were fed a control (CSAA) diet, CDAA+1% CHOL (CDAA/c) diet, or CDAA/c diet supplemented with NTZ 100 mg/kg/day for 12 weeks. After sacrifice, 4-HNE levels were determined by Immunochemistry and quantified (panel A). Representative images of 4-HNE staining for each group are shown on panel B (Magnification ×300).

FIG. 2: chronic oral administration of NTZ induces the hepatic expression of GSTA1 (A) and GSTA2 (B) at the mRNA levels.

6 week-old C57BL/6 mice were fed a control (CSAA) diet, CDAA+1% CHOL (CDAA/c) diet, or CDAA/c diet supplemented with NTZ 100 mg/kg/day for 12 weeks. After the sacrifice, the hepatic levels of hepatic GSTa mRNA were analyzed by RNAseq and the count levels were determined.

FIG. 3: chronic oral administration of NTZ induces the hepatic expression of GSTA4 at the mRNA levels.

6 week-old C57BL/6 mice were fed a control (CSAA) diet, CDAA+1% CHOL (CDAA/c) diet, or CDAA/c diet supplemented with NTZ 100 mg/kg/day for 12 weeks. After the sacrifice, the hepatic levels of GSTA4 mRNA were analysed by RNAseq and the normalized count levels were determined.

FIG. 4: Genes differentially induced by NTZ are significantly enriched in Nrf2 target genes. 6 week-old C57BL/6 mice were fed a control (CSAA) diet, CDAA+1% CHOL (CDAA/c) diet, or CDAA/c diet supplemented with NTZ 100 mg/kg/day for 12 weeks. After the sacrifice, transcriptome was analyzed by RNAseq. The proportion of Nrf2 target genes in the whole RNA-seq identified transcriptome (27636 genes) was calculated and compared to the proportion of Nrf2 target genes in the subset of genes differentially expressed in the NTZ+CDAA/c vs CDAA/c conditions.

FIG. 5: TZ induces Nrf2-ARE (Antioxidant Response Element) signaling in human hepatocytes. Antioxidant response element (ARE)-mediated luciferase activity was measured in HepG2 cells treated with TZ. DL-Sulforaphane (DLS) was used as a reference compound.

FIG. 6: TZ prevents glutathione (GSH) depletion in human hepatocytes exposed to Menadione, an oxidative stress inducer.

Intracellular GSH level was monitored by a thiol tracker (ThiolTracker Violet dye) in menadione-stressed hepatocytes in either presence or absence of TZ. N-Acetylcysteine (NAC) was used as a positive control.

### EXAMPLES

### Evaluation of Nitazoxanide in a Chronic CDAA+1% Cholesterol Model of Fibrosing NASH (12 Weeks)

Experimental Design

Given the prominent role of oxidative stress in NASH pathogenesis, we evaluated NTZ capacity to prevent redox homeostasis dysregulation in the CDAA/c diet induced NASH model.

The choline-deficient and L-amino acid-defined (CDAA) diet lacks choline, which is essential for hepatic β-oxidation and very low density lipoprotein production, and is believed to induce hepatocellular steatosis. Subsequently, lipid peroxidation and oxidative stress lead to lobular inflammation, comprehensively resulting in fibrosis.

In the current study, the preventive effects of NTZ 100 mg/kg/day were assessed in a murine model. 6 week-old male C57Bl/6J mice were fed a control (CSAA) diet (n=8), CDAA+1% cholesterol diet (n=12), or CDAA+1% cholesterol diet supplemented with NTZ 100 mg/kg/day (n=8) for 12 weeks. The food was purchased from Ssniff® company (Soest, Germany). Nitazoxanide (Interchim, Ref #RQ550) was incorporated by Ssniff® into CDAA+1% chol diet in powder form to the required dose.

The body weight and the food intake were monitored twice per week. On the last day of treatment, mice were sacrificed after a 6 h fasting period. The liver was rapidly excised for transcriptomic and histological studies.

All animal procedures were performed according to standard protocols and in accordance with the standard recommendations for the proper care and use of laboratory animals.

Transcriptomic Studies

RNA Extraction

Hepatic Total RNA was isolated using Nucleospin® 96 Kit (Macherey Nagel) following manufacturer's instructions. 150 ng of total RNA were reverse transcribed in cDNA using M-MLV-RT (Moloney Murine Leukemia Virus Reverse Transcriptase) (Invitrogen cat #28025) in presence of RT buffer 1× (Invitrogen cat #P/NY02321), 1 mM DTT (Invitrogen cat #P/NY00147), 0.5 mM dNTPs (Promega), 200 ng pdN6 (Roche cat #11034731001) and 40 U of Ribonuclease inhibitor (Promega cat #N2515).

RNA-Sequencing:

Upon measurement of RNA samples concentration by nanodrop, the quality was assessed using bioanalyser. Libraries were prepared using the Illumina TruSeq stranded mRNA LT kit and mRNA were sequenced using a NextSeq 500 device (paired-end sequence, 2×75 bp), with a High Output flow cell.

RNA-Seq Data Analysis:

Reads were cleaned using Trimmomatic v.0.36 with the following parameters: SLIDINGWINDOW:5:20 LEADING:30 TRAILING:30 MINLEN:60.Then reads were aligned on the genome reference (Mus musculus GRCm38.90) with rnacocktail using hisat2 v.2.1.0 as aligner with default parameters.

A count table was produced using featureCounts v1.5.3 with default parameters.

To identify differentially expressed genes (DE genes), we used R (version 3.4.3) and the DESEa2 library (v. 1.18.1). Gene annotations were retrieved using the AnnotationDbi library (v. 1.40.0). Briefly, the count matrix produced by FeatureCounts was analyzed by the DESeqDataSetFromMatrixO function followed by the DEseq( ) function from the DESeq2 library. For each condition (ie comparison NTZ+CDAA/c vs CDAA/c), the fold change and the p-value were retrieved using the results( ) function from DESeq2. The different tables were merged using the Ensembl ID as a key.

To assess the role of Nrf2 on the transcriptome remodeling induced by NTZ, a table listing Nrf2 target genes was produced by merging the information from Hayes and McMahon and, Jung and Kwak (Hayes and McMahon 2009; Jung and Kwak 2010), the mouse Nfe212 targets from TRRUST database (https://www.grnpedia.org/trrust/), the NRF2 pathway from Wikipathway (https://www.wikipathways.org/index.php/Pathway:WP2884) and the ChIP-seq data from Chorley et al. (Chorley, Campbell et al. 2012). This Nrf2 target genes list was used to identify Nrf2 target genes in the whole RNA-seq identified transcriptome (27636 genes) or in the set of genes differentially expressed in the NTZ+CDAA/c vs CDAA/c conditions (is considered as a differentially expressed a gene meeting the following criteria: at least |fold change vs CDAA/c|>1.4 fold and an adjusted p-value <0.01). The ratio between the whole pool of genes and the Nrf2 target genes was calculated and expressed as percent. To decipher if the proportion in Nrf2 target genes observed in the NTZ+CDAA/c vs CDAA/c condition differs from the proportion observed in the whole identified transcriptome, a Chi-square test was performed.

Histology

At sacrifice, liver samples were processed for histological analysis and examined as follows.

Tissue Embedding and Sectioning

The liver slices were first fixed for 40 hours in formalin 4% solution followed by several dehydration steps in ethanol (successive baths at 70, 80, 95 and 100% ethanol). The liver pieces were subsequently incubated in three xylene baths followed by two baths in liquid paraffin (58° C.). Liver pieces were then put into small racks that were gently filled with Histowax® to completely cover the tissue. Then, tissue samples were thicked in 3 μm sections. Sections were prepared for immunohistochemistry (IHC).

Immunohistochemistry Assay: 4-HNE (4-Hydroxynonenal)

Immunohistochemistry assay was performed by using an immunoperoxidase protocol. Sections were dewaxed at 58° C. and in xylene baths (2×3 min). The specimens were hydrated with ethanol (successive baths at 100%, 100%, 95% and 70%) (3 min each) and submerged in 1×PBS (2×5 min). Subsequently, endogenous peroxidase was blocked with H₂O₂ solution (0.3% H₂O₂ in distilled water) for 30 min, followed by three washes in 1×PBS for 5 min. Furthermore, heat mediated antigen retrieval was performed with citrate buffer at pH 6.0 for 40 min at 95° C. To block nonspecific binding, 1×PBS solution with 3% normal goat serum and 0.1% Triton was added for 60 min. Subsequently, the tissues were incubated with primary 4-HNE antibody overnight at 4° C. and rinsed with 1×PBS (3×5 min). The tissues were incubated with HRP secondary antibody for 1 h at room temperature and then rinsed with 1×PBS (3×5 min). Slides are then revelated with the peroxidase substrate 3,3′-diaminobenzidine ((DAB) for 15 min, and rinsed with tap water. Finally, the stains were counterstained with Mayer hematoxylin for 3 min and rinsed with tap water (2 min) and tissues were dehydrated in ethanol and xylene.

4-HNE IHC Analysis:

The histological examinations and scoring were performed blindly. Images were acquired using Pannoramic 250 Flash II digital slide scanner (3DHistech). Scoring: seven randomly selected fields from each section were examined and analyzed in QuantCenter software. 4-HNE accumulation was calculated as 4-HNE-positive area/total selected fields area.

Intracellular GSH Detection:

Tizoxanide (Interchim; cat #RP253) was dissolved in DMF (Sigma; cat #227056). Menadione (Sigma; cat #M2518) and N-Acetylcysteine were dissolved (Sigma; cat #A9165) in water.

Hep G2 cells (ECACC) were plated at a density of 20 000 cells par well into a 96-well microplate in 100 μl of DMEM (Gibco; cat #41965-039) supplemented with 1% P/S (Gibco; cat #15140-122), 1% glutamine(Gibco; cat #25030-024).The complete medium contained 10% SVF (Gibco; cat #10270-106). The next day, culture medium was removed and replaced by 100 μL of DMEM (Gibco; cat #31053-028) without Red Phenol and SVF but supplemented with 1% P/S (Gibco; cat #15140-122), 1% glutamine(Gibco; cat #25030-024). The serum deprived HepG2 were preincubated for 1 hour with TZ or N-Acetylcysteine (NAC) then exposed to Menadione (MND) for 2 hours. Compounds were dissolved in their respective vehicles a mentioned above and diluted in the deprived culture medium. 5 μl of dilution of 20× dilution were added on cells to reach a final concentration of 1 μM for TZ, 10 mM of NAC and 100 μM of MND respectively. The cellular level of reduced glutathione was subsequently monitored using the ThiolTracker Violet dye (Invitrogen, cat #T10096). Briefly, cells were washed two times with DPBS (Invitrogen, Cat #14287-080) then incubated for 30 min at 37° with 100 μl of prewarmed ThiolTracker Violet dye solution prepared in DPBS according the supplier 's instructions and fluorescence was measured (Ex:404 nm, Em: 526 nm)

ARE Reporter—HepG2 Cell Line

ARE Reporter—Hep G2 cells(BPS Bioscience, Inc., San Diego cat #60513) were cultured following manufacturer's instructions. After thawing (BPS thaw medium 1, cat #60187), cells were cultured in growth-medium (BPS growth medium 1K, cat #79533) and subsequently plated at a density of 40 000 cells par well in a 96-well microplate in 45 μl of assay medium (BPS thaw medium). TZ (Interchim; cat #RP253) & DL-Sulforaphane (Sigma; cat #S4441) were dissolved in DMSO and diluted into assay medium. 5 μl of 10× dilution were added on cells to reach a final concentration of 1 μM and 3 μM for TZ and DL-Sulforaphane (DL-Sulfo) respectively. After 18 h exposure, luciferase activity was determined. 50 μl of One-Step Luciferase assay system (BPS cat #60690) were added per well and after −15 min of rocking at room temperature, luminescence was measured using a luminometer.

Statistical Analysis

Experimental results were expressed as mean±SEM and plotted as bar graphs. Statistical analyses were performed using Prism Version 7, as follows:

In Vivo Data

CSAA vs CDAA+1% chol groups were compared by a Student t-test (#: p<0.05; ##: p<0.01; ###: p<0.001) or by a Mann-Whitney test ($: p<0.05; $$: p<0.01; $$$: p<0.001).

NTZ treated group was compared to CDAA+1% chol diet by by a Student t-test (#: p<0.05; ##: p<0.01; ###: p<0.001) or by a Mann-Whitney test ($: p<0.05; $$: p<0.01; $$$: p<0.001).

In Vitro Data

For ARE-reporter luc assay, each treatment effect was compared to vehicle effect by a Student t-test (#: p<0.05; ##: p<0.01; ###: p<0.001).

For Intracellular GSH detection, Menadione condition was compared to unstimulated condition by a Mann-Whitney test ($: p<0.05; $$: p<0.01; $$$: p<0.001). Each treatment effect was compared to vehicle effect by a Student t-test (#: p<0.05; ##: p<0.01; ###: p<0.001) or by a Mann-Whitney test ($: p<0.05; $$: p<0.01; $$$: p<0.001).

Results

Oxidative stress is an important pathophysiological mechanism of NASH (Koruk, Taysi et al. 2004; Masarone, Rosato et al. 2018) and it is widely recognized that 4-HNE, a peroxidized aldehyde product of unsaturated fatty acids, is an indicator of oxidative stress (Takeuchi-Yorimoto, Noto et al. 2013).

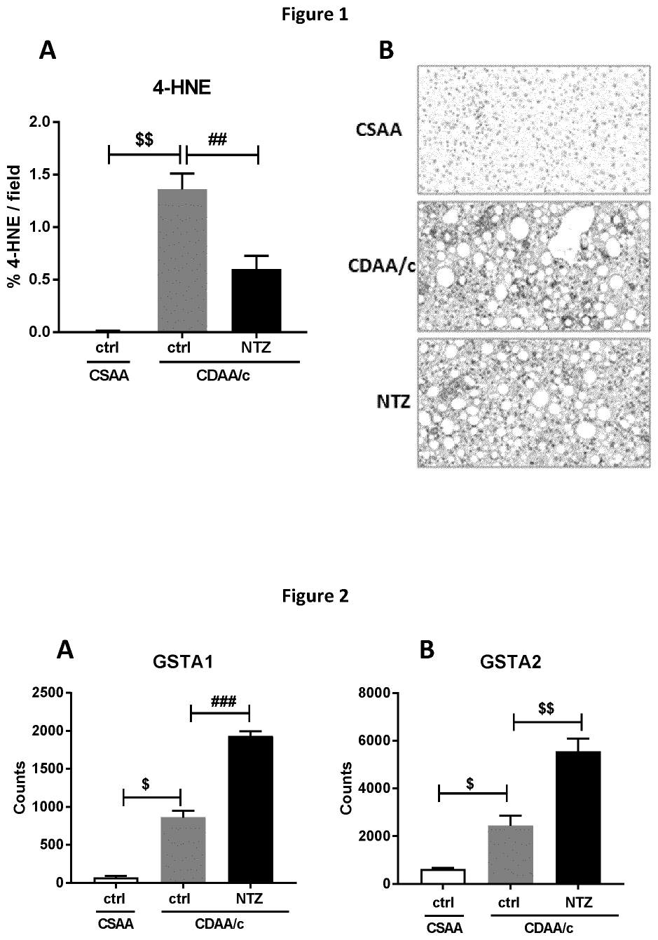

Accordingly, a significant increase of intrahepatic 4-HNE levels is observed in the CDAA/c group in comparison with the CSAA diet as shown in FIG. 1 whilst the levels of 4-HNE in the group that was exposed to NTZ in parallel of the CDAA/c regimen is significantly lower, indicating a protective effect of NTZ against oxidative stress-induced lipid peroxidation.

To further investigate the anti-oxidative stress effect of NTZ, transcriptomic analyses were conducted on liver samples. As shown in FIG. 2, the levels of hepatic GSTA1 transcripts (panel A) as well as the levels of GSTA2 (panel B) transcripts are significantly induced in the CDAA/c group in comparison with the CSAA group reflecting the implementation of an antioxidant defense mechanism. Interestingly a significant induction of the expression is observed for both enzymes comparing the group which received NTZ+CDAA/c versus the CDAA/c regimen alone suggesting an improvement of the defense against oxidative stress.

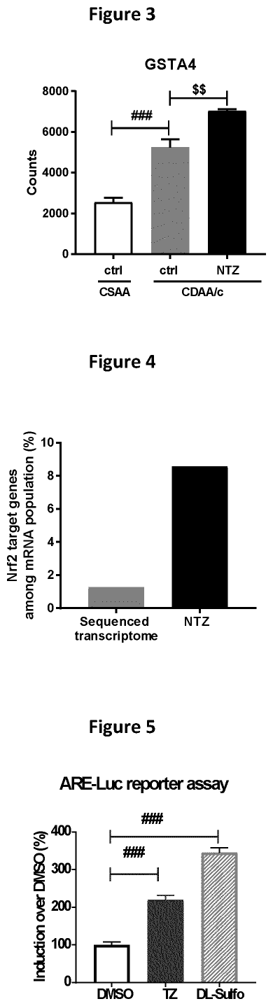

Indeed, GSTA are well known as detoxification enzymes allowing the elimination of HNE by conjugation with glutathione. The levels of hepatic GSTA4 mRNA were also analyzed (FIG. 3) since this enzyme is considered as a key player for HNE detoxification with higher activity for conjugating HNE to GSH comparatively to other isoenzymes (Awasthi, Ramana et al. 2017). As for the other GSTs, a significant induction of the GSTA4 transcript level is observed comparing the CDAA/c group with the CSAA group and mice fed with the NTZ+CDAA/c regimen revealed a significant higher level of GSTA4 in comparison with the CDAA/c group comforting the finding that NTZ promotes a defense mechanism against oxidative stress.

In mice and humans, GSTA1, GSTA2 and GSTA4 are described as genes positively regulated by Nrf2, a key regulator of cellular redox status (Hayes and Dinkova-Kostova 2014). Accordingly, we compared the proportion of Nrf2 target genes observed in the whole sequenced transcriptome with the proportion of Nrf2 genes among the genes induced by NTZ treatment (obtained by comparison of NTZ+CDAA/c signature vs CDAA/c conditions). Unexpectedly, a significant enrichment of the Nrf2 signature was induced by NTZ treatment (FIG. 4). Whereas Nrf2 target genes represent 1.3% of the whole identified transcriptome in the RNA-seq data, Nrf2-regulated genes was found to constitute more than 8% of all differentially expressed genes in mice treated with NTZ.

To confirm the potency of NTZ to activate the Nrf2 antioxidant pathway at a functional level, Antioxidant Response Element (ARE)-mediated luciferase activity was measured in HepG2 cells treated with TZ (the active metabolite of NTZ). Under basal conditions, Nrf2 is retained in the cytosol by binding to the cytoskeletal protein Keap1 and upon exposure to oxidative stress or other ARE activators, Nrf2 is released from Keap1 and translocates to the nucleus, where it can bind to the ARE, leading to the expression of antioxidant and phase II enzymes that protect the cell from oxidative damage. As shown in FIG. 5, TZ exposure leads to a significant increase of ARE-mediated transcription reflecting its capacity to induce Nrf2 translocation to the nucleus and the associated ARE signaling in human hepatocytes. To summarize, these data indicate that NTZ can induce a mechanism of defense against oxidative stress and that part of the underlying mechanism relies on Nrf2.

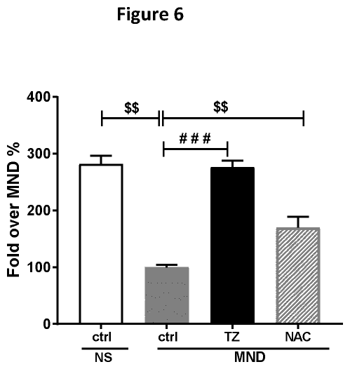

In complement of these analyses, we evaluated the impact of TZ on GSH (reduced Glutathione) content, which is the most powerful antioxidant in the liver (Al-Busafi, Bhat et al. 2012), in hepatocytes exposed to menadione (MND), an oxidative stress inducer. As shown in FIG. 6, MND exposure induced as expected, a significant decrease of cellular GSH. TZ treatment totally prevented this MND-induced reduction. This result confirms the antioxidant properties TZ, the active metabolite of NTZ.

In conclusion, all these results demonstrate that NTZ and/or its metabolite TZ can provide therapeutic benefits in disease associated with oxidative stress.

### REFERENCES

Al-Busafi, S. A., M. Bhat, et al. (2012). “Antioxidant therapy in nonalcoholic steatohepatitis.” Hepatitis research and treatment 2012: 947575-947575.
Awasthi, Y. C., K. V. Ramana, et al. (2017). “Regulatory roles of glutathione-S-transferases and 4-hydroxynonenal in stress-mediated signaling and toxicity.” Free Radic Biol Med 111: 235-243.
Chorley, B. N., M. R. Campbell, et al. (2012). “Identification of novel NRF2-regulated genes by ChIP-Seq: influence on retinoid X receptor alpha.” Nucleic Acids Res 40(15): 7416-7429.
de AraGjo, R., D. Martins, et al. (2016). “Oxidative Stress and Disease—from the edited volume: A Master Regulator of Oxidative Stress—The Transcription Factor Nrf2, Edited by Jose Antonio Morales-Gonzalez, Angel Morales-Gonzalez and Eduardo Osiris Madrigal-Santillan.”
de Carvalho, L. P. S., C. M. Darby, et al. (2011). “Nitazoxanide disrupts membrane potential and intrabacterial pH homeostasis of Mycobacterium tuberculosis.” ACS Med. Chem. Lett. 2(Copyright (C) 2015 American Chemical Society (ACS). All Rights Reserved.): 849-854.
Di Santo, N. and J. Ehrisman (2014). “A functional perspective of nitazoxanide as a potential anticancer drug.” Mutat. Res., Fundam. Mol. Mech. Mutagen. 768(Copyright (C) 2015 American Chemical Society (ACS). All Rights Reserved.): 16-21.
Hayes, J. D. and A. T. Dinkova-Kostova (2014). “The Nrf2 regulatory network provides an interface between redox and intermediary metabolism.” Trends Biochem Sci 39(4): 199-218.
Hayes, J. D. and M. McMahon (2009). “NRF2 and KEAP1 mutations: permanent activation of an adaptive response in cancer.” Trends Biochem Sci 34(4): 176-188.
Hoffman, P. S., G. Sisson, et al. (2007). “Antiparasitic drug nitazoxanide inhibits the pyruvate oxidoreductases of Helicobacter pylori, selected anaerobic bacteria and parasites, and Campylobacter jejuni.” Antimicrob. Agents Chemother. 51(Copyright (C) 2015 American Chemical Society (ACS). All Rights Reserved.): 868-876.
Jung, K. A. and M. K. Kwak (2010). “The Nrf2 system as a potential target for the development of indirect antioxidants.” Molecules 15(10): 7266-7291.
Koruk, M., S. Taysi, et al. (2004). “Oxidative stress and enzymatic antioxidant status in patients with nonalcoholic steatohepatitis.” Ann Clin Lab Sci 34(1): 57-62.
Masarone, M., V. Rosato, et al. (2018). “Role of Oxidative Stress in Pathophysiology of Nonalcoholic Fatty Liver Disease.” Oxid Med Cell Longev 2018: 9547613.
Rossignol, J.-F. (2014). “Nitazoxanide: A first-in-class broad-spectrum antiviral agent.” Antiviral Res. 110(Copyright (C) 2015 American Chemical Society (ACS). All Rights Reserved.): 94-103.
Rossignol, J. F. and R. Cavier (1975). DE2438037A1-2-Benzamido-5-nitrothiazoles, S. P. R. L. Phavic, Belg. 11 pp.
Takeuchi-Yorimoto, A., T. Noto, et al. (2013). “Persistent fibrosis in the liver of choline-deficient and iron-supplemented L-amino acid-defined diet-induced nonalcoholic steatohepatitis rat due to continuing oxidative stress after choline supplementation.” Toxicol Appl Pharmacol 268(3): 264-277.
Wuts, P. G. M. and T. W. Greene (2007). Greene's Protective Groups in Organic Synthesis, Fourth Edition, John Wiley & Sons.

1. A method for treating oxidative stress associated with a disease, the method comprising administering to a subject a compound of formula (I) or a pharmaceutical acceptable salt thereof, 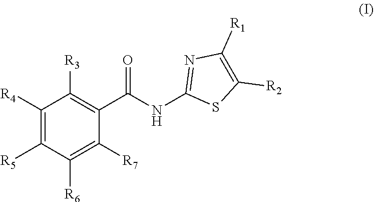
in which:
R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group (NO2), an amino group (NH2), a (C1-C6)alkylamino group, an amido group, a (C1-C6)alkylamido group or a (C1-C6)dialkylamido group;
R2 represents a hydrogen atom, a deuterium atom, a NO2 group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH₂ group or a (C1-C6)alkylamino group;
or R1 and R2, together with the carbon atoms to which they are attached, form a substituted or unsubstituted 5- to 8-membered cycloalkyl, heterocyclic or aryl group;
R3, R4, R5, R6, and R7, identical or different, represent a hydrogen atom, a deuterium atom, a halogen atom, a hydroxyl group, a (C1-C6)alkylcarbonyl group, an (C1-C6)alkyl group, an (C1-C6)alkyloxy group, an (C1-C6)alkylthio group, an (C1-C6)alkylcarbonyloxy group, an (C6-C14)aryloxy group, a (C6-C14)aryl group, a heterocyclic group, a (C3-C14)cycloalkyl group, a NO2 group, a sulfonylaminoalkyle group, an NH2 group, an amino(C1-C6)alkyl group, an (C1-C6)alkylcarbonylamino group, a carboxylic group, a carboxylate group, or a R9 group;
R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A): 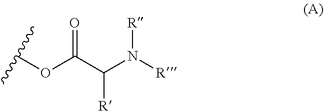
wherein R′ represents an (C1-C6)alkyl group, an (C2-C6)alkenyl group, an (C2-C6)alkynyl group, a (C3-C14)cycloalkyl group, (C3-C14)cycloalkylalkyl group, a (C3-C14)cycloalkyl(C2-C6)alkenyl group, a (C3-C14)cycloalkenyl group, a (C3-C14)cycloalkenyl(C1-C6)alkyl group, a (C3-C14)cycloalkenyl(C2-C6)alkenyl group or a (C3-C14)cycloalkenyl(C2-C6)alkynyl group; wherein R″ and R′″, independently, represent a hydrogen atom, an (C1-C6)alkyl group, or a nitrogen protecting group; and
R8 represents a hydrogen atom, a deuterium atom, a glucuronidyl group, or a 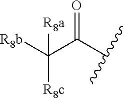
group wherein, R8a, R8b and R8c, identical or different, represent a hydrogen atom or a deuterium atom.

2. The method according to claim 1, wherein the disease is selected from the group consisting of a neurological disorder, a metabolic condition, a cardiovascular disease, cataract, atherosclerosis, ischemia, ischemic brain damage, lung ischemia-reperfusion injury, scleroderma, stroke, inflammation, rheumatoid arthritis, a respiratory disease, an autoimmune disease, a liver disease, a kidney disease, a skin condition, an infection and cancer.

3. A method for treating a disease in which oxidative stress is involved, the method comprising administering to a subject a compound of formula (I) or a pharmaceutically acceptable salt thereof: 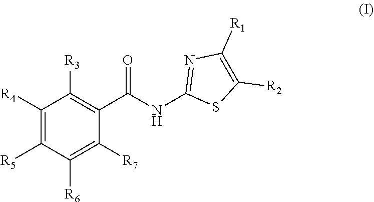
in which:
R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group (NO2), an amino group (NH2), a (C1-C6)alkylamino group, an amido group, a (C1-C6)alkylamido group or a (C1-C6)dialkylamido group;
R2 represents a hydrogen atom, a deuterium atom, a NO2 group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH₂ group or a (C1-C6)alkylamino group;
or R1 and R2, together with the carbon atoms to which they are attached, form a substituted or unsubstituted 5- to 8-membered cycloalkyl, heterocyclic or aryl group;
R3, R4, R5, R6, and R7, identical or different, represent a hydrogen atom, a deuterium atom, a halogen atom, a hydroxyl group, a (C1-C6)alkylcarbonyl group, an (C1-C6)alkyl group, an (C1-C6)alkyloxy group, an (C1-C6)alkylthio group, an (C1-C6)alkylcarbonyloxy group, an (C6-C14)aryloxy group, a (C6-C14)aryl group, a heterocyclic group, a (C3-C14)cycloalkyl group, a NO2 group, a sulfonylaminoalkyle group, an NH2 group, an amino(C1-C6)alkyl group, an (C1-C6)alkylcarbonylamino group, a carboxylic group, a carboxylate group, or a R9 group;
R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A): 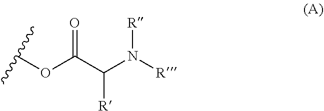
wherein R′ represents an (C1-C6)alkyl group, an (C2-C6)alkenyl group, an (C2-C6)alkynyl group, a (C3-C14)cycloalkyl group, (C3-C14)cycloalkylalkyl group, a (C3-C14)cycloalkyl(C2-C6)alkenyl group, a (C3-C14)cycloalkenyl group, a (C3-C14)cycloalkenyl(C1-C6)alkyl group, a (C3-C14)cycloalkenyl(C2-C6)alkenyl group or a (C3-C14)cycloalkenyl(C2-C6)alkynyl group; wherein R″ and R′″, independently, represent a hydrogen atom, an (C1-C6)alkyl group, or a nitrogen protecting group; and
R8 represents a hydrogen atom, a deuterium atom, a glucuronidyl group, or a 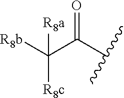
group wherein, R8a, R8b and R8c, identical or different, represent a hydrogen atom or a deuterium atom.

4. The method according to claim 3, wherein the disease is selected from the group consisting of a neurological disorder, a metabolic condition, a cardiovascular disease, cataract, atherosclerosis, ischemia, ischemic brain damage, lung ischemia-reperfusion injury, scleroderma, stroke, inflammation, rheumatoid arthritis, a respiratory disease, an autoimmune disease, a kidney disease and a skin condition.

5. The method according to claim 3, wherein the disease is selected from the group consisting of Alzheimer's disease, Parkinson's disease, Huntington's disease, tardive dyskinesia, epilepsy, an acute disease of the central nervous system, obesity, insulin resistance, dyslipidemia, impaired glucose tolerance, high blood pressure, atherosclerosis, diabetes, metabolic syndrome, myocardial ischemia, ischemic brain damage, lung ischemia-reperfusion injury, scleroderma, stroke, inflammatory bowel disease and rheumatoid arthritis.

6. The method according to claim 1, wherein said compound is selected from the group consisting of nitazoxanide, tizoxanide and a pharmaceutically acceptable salt thereof.

7. The method according to claim 1, wherein said compound is NTZ or a pharmaceutically acceptable salt thereof.

8. The method according to claim 1, wherein the compound is formulated for oral administration.

9. The method according to claim 1, wherein the compound is formulated in a pill, a tablet or a suspension for oral ingestion.

10. The method of claim 2, wherein the neurological disorder is a central nervous system disorder.

11. The method of claim 2, wherein the ischemia is myocardial ischemia.

12. The method of claim 2, wherein the inflammation is inflammatory bowel disease.

13. The method of claim 4, wherein the neurological disorder is a central nervous system disorder.

14. The method of claim 4, wherein the ischemia is myocardial ischemia.

15. The method of claim 4, wherein the inflammation is inflammatory bowel disease.

16. The method of claim 5, wherein the acute disease of the central nervous system is a spinal cord injury and/or brain trauma.

17. The method of claim 5, wherein the diabetes is type 1 or type 2 diabetes.
